(19) United States
(12) Patent Application Publication
(10) Pub. No.: US 2010/0228300 A1
(43) Pub. Date: Sep. 9, 2010

(54) Radiosurgery Compatible Bone Anchor

(76) Inventors: Kevin Armstrong, Irvine, CA (US); Thomas Chen, Le Canada, CA (US); Thomas Meiser, Denver, CO (US)

Correspondence Address: Atlantic Coast Patent Group, LLC, P.O Drawer 551375, Ft. Lauderdale, FL, 33355, US

(21) Appl. No.: 12/697,500

(22) Filed: Feb. 1, 2010

Related U.S. Application Data
(60) Provisional application No. 61/149,044, filed on Feb. 2, 2009.

(51) Int. Cl. A61B 17/86

(52) U.S. Cl. 606/301

(57) ABSTRACT

There is provided an anchoring mechanism/bone anchor comprised of a radiotranslucent material for securing vertebral stabilizing implants to vertebral bone. The anchoring mechanism is preferably a pedicle screw comprised of a fiber material embedded in a radiolucent matrix material.

### CROSS-REFERENCE TO RELATED APPLICATION

This application claims the benefit under 35 U.S.C. §119(e) of U.S. Provisional Application Ser. No. 61/149,044, filed Feb. 2, 2009, which is hereby incorporated in its entirety by reference thereto.

### FIELD OF THE INVENTION

The present invention relates to radiosurgery compatible implants for immobilizing adjacent vertebral segments and more particularly the anchoring mechanisms used to secure these implants to vertebral bone.

### BACKGROUND OF THE INVENTION

Various spinal stabilization methodologies are known in the field of medicine for the immobilization of adjacent vertebral segments of the spine in order to treat certain spinal traumas and pathologies. These methodologies typically involve the surgical placement of a vertebral stabilizing device or assembly such as a plate, cage, rod system, etc. that is positioned longitudinally along those spinal units to be fused and attached thereto using one or more “bone anchors” that are embedded into the vertebral bone.

These bone anchors typically consist of an anchoring portion that is surgically embedded in the bone and a receiving or head portion designed to contact and receive the stabilizing device by way of a static or dynamic coupling, either directly or indirectly via a coupling mechanism.

The most commonly used bone anchor is the pedicle screw that is commercially available in numerous designs and sizes. For example, U.S. Pat. No. 7,445,627 to Hawkes discloses a polyaxial pedicle screw while U.S. Pat. No. 6,368,319 to Schaefer discloses a monoaxial pedicle screw having a specialized safety anchoring mechanism. Examples of other various pedicle screws are shown in U.S. Pat. Nos. 7,445,627, 7,163,539, 6,858,030, 6,840,940, 6,565,567, 6,554,834, 6,488,681, 6,485,494, 6,402,752, 6,368,319, 6,183,472, 6,063,089, 5,725,528, 5,207,678, 4,946,458, and 4,887,596. These pedicle screws all comprise a threaded anchoring portion that is surgically embedded into vertebral bone.

A major consideration in the design of an effective bone anchor concerns the capability of the anchor to withstand adequate load-bearing stresses transferred to it from a vertebral stabilizing device once the device has been implanted in a patient. Tremendous compressive and shear forces are transferred from the device into the anchor that has been surgically embedded in vertebral bone. The industry standard anchors currently used by surgeons are pedicle screws constructed of extremely durable materials such as stainless steel, titanium and titanium alloys that can withstand such forces.

Another major consideration in the design of an effective bone anchor such as a pedicle screw concerns the resulting background radiation scatter emitted from the screw when exposed to diagnostic imaging or radiosurgery treatments. In the past, pedicle screws were manufactured from elemental metals or metal alloys that rendered an unfavorable level of interference/radiation scatter when exposed to post-surgical imaging studies by CT and especially, MRI. This rendered a problematic visual obstruction on the films that compromised radiological evaluation not to mention creating even more serious issues for radiosurgery techniques. The problem was eventually somewhat addressed by the use of stainless steel, titanium and titanium alloys such as Nitinol (a titanium/nickel alloy) that had a much lower incidence of interference/radiation scatter.

Importantly, there are instances where patients with advanced vertebral tumors require surgical removal of the tumor in a way that destabilizes the spine. In such cases, the surgeon must place pedicle screws in the spine to attach a device for stabilizing the spine. Surgeons typically employ existing pedicle screw technology, namely the industry standard polyaxial pedicle screw made from stainless steel, titanium alloy or commercially pure titanium, that is not necessarily well suited for this specific application where subsequent therapies might be warranted.

For example, the treatment team will often prescribe radiosurgery in an attempt to eradicate remaining tumor cells. Radiosurgery is a computer driven robotic system that focuses a radiation beam at the tumor from different angles. Radiosurgery requires CT and MRI scans of the patient's spine to allow the surgeon and radiologist to visualize the borders of the tumor in relation to the spinal anatomy (spinal cord, vertebral body, pedicles, etc.) so they can program the robot to “shoot” the tumor from the different angles while avoiding neural structures.

Unfortunately, the materials used to manufacture the industry standard pedicle screws or other bone anchors still retain an undesirable level of radiopacity as well as the problematic issue of radiation scatter in post surgical irradiation of spinal tumors, a particularly intricate radiosurgery procedure due to the proximity of the tumor to the spinal cord to the bony vertebral structures housing these screws.

The aforementioned problems have recently become even more troublesome with the advent of new and improved radiosurgery techniques such as that afforded by Cyberknife® technology where the accuracy of the treatment beam has become so precise that what once may have been an acceptable level of scatter and radiopacity has now become clearly unacceptable and problematic.

One of the leading radiosurgery systems is the Cyberknife® technology which utilizes a frameless robotic radiosurgery system invented by John R. Adler, a Stanford University Professor of Neurosurgery and Radiation Oncology. A major hallmark of Cyberknife® technology is that the radiation is produced from a small linear particle accelerator coupled with a complex computer-guided robotic arm which allows the radiation beam to be directed at any part of the body from any direction in a highly precise manner. The image guidance system is another unique component in the Cyberknife® system. X-ray or CT imaging cameras are located on supports around the patient affording instantaneous or real time images of the irradiation site to be obtained

For a tumor located in the spine, the imaging system uses the internal anatomy to directly track tumor borders with extreme precision and eliminates the need for external frames or implanted fiducials. It registers anatomical landmarks to track, detect, and compensate for any movements of the spine using real-time tracking throughout the treatment. Accordingly, the system affords the delivery of high doses of radiation with sub-millimeter accuracy while avoiding damage to healthy tissue.

The advent of such highly precise irradiation systems has precipitated the need for new and improved radiotranslucent materials to be incorporated into implantable devices in order to eliminate or further reduce any possibility of radiological image obstruction or radiation scatter during radiosurgery therapy. This is especially true for spinal implant technologies where radiation scatter from radiosurgery could likely result in irreversible tissue damage to the spinal cord. In fact, the aforementioned systems have become so precise that the materials currently in use for spinal implant devices have become one of the precision limiting factors in the accurate delivery of the radiation dose due to the radiopacity and radiation scattter issues associated therewith.

This dilemma becomes even more critical regarding the bone anchoring mechanisms for these devices due to the proximity of the anchoring site to the spinal cord. Since these anchors must be embedded into vertebral bone that is dangerously close to the spinal nerves, the elimination of undesirable radiopacity and radiation scatter becomes of even greater concern to the physician with respect to such anchors.

Additionally, bone anchors are also required to endure tremendous compressive and shear forces transferred to them from the spine once the device is implanted in a patient. As such, they must be fashioned from a material strong enough to withstand these forces while simultaneously achieving acceptable levels of radiotranslucency and radiation scatter so as not to become a precision limiting factor in the advanced radiosurgery techniques available today.

Accordingly, it would be advantageous for the medical profession to have access to a new and improved anchoring mechanism such as a pedicle screw that eliminates unacceptable levels of radiotranslucency and radiation scatter while withstanding the compressive stresses mentioned above.

Moreover, it would also be advantageous to provide a new anchoring mechanism and method for attaching a vertebral stabilizing device to patients being treated for spinal tumors using radiotherapy.

### SUMMARY OF THE INVENTION

In accordance with one aspect of the invention, there is provided an anchoring mechanism/orthopedic anchor comprised of a radiotranslucent material for securing vertebral stabilizing devices to vertebral bone. The anchoring mechanism includes an anchoring portion that is suitable to be embedded into vertebral bone as well as a head portion that can be coupled directly or indirectly to the stabilizing device. For instances of indirect coupling, a separate coupling mechanism such as a “saddle”, “tulip” or other connector is employed to communicate with and receive some structural aspect of the stabilizing device be it a rod, rod assembly, plate, cage, etc. In one preferred, exemplary embodiment, the bone anchor of the present invention is fashioned in the form of a pedicle screw having any one of the many configurations set forth in the prior art, some of those being referenced below in the detailed description of the invention.

As mentioned above, the bone anchoring mechanism of the present invention includes an anchoring portion that is suitable to be embedded into vertebral bone as well as a head portion that can be coupled directly or indirectly to the stabilizing device. In one embodiment of the present invention, the entire anchor (both anchor portion and head portion) is formed from a radiotranslucent material. In an alternative embodiment, only the anchoring portion is formed from the radiotranslucent material while the head portion is fashioned from stainless steel, titanium, titanium alloy or other appropriate material.

The radiotranslucent material preferably comprises a medical grade, radiotranslucent polymer or co-polymer that is capable of forming a rigid, loadbearing matrix sufficient to handle both shear and compressive forces transferred to it by a vertebral stabilizing device that has been affixed to a patient's spine. In another exemplary embodiment, the radiotranslucent material comprises a composite substance that is in turn comprised of a medical grade fiber material dispersed within the aforementioned polymeric matrix.

In yet a further embodiment, the radiotranslucent polymer or copolymer is comprised of at least one of the polymeric materials selected from the following group: poly-ether-ether-ketone (PEEK), poly-ether-ketone-ketone (PEKK), poly-methylmethacrylate (PMMA), polysulfone, polylactide (PLA), poly-L-lactide (PLLA), and poly(glycolic acid) (PGA); and, more particularly, a ketone-based polymer such as poly-ether-ether-ketone (PEEK) or poly-ether-ketone-ketone (PEKK); and, most particularly, poly-ether-ether-ketone (PEEK).

In another embodiment, the fiber material is comprised of a least one of either carbon fibers or polyamide fibers, and more particularly, is substantially comprised of short strand carbon fibers. In yet another exemplary embodiment, the head portion of the pedicle screw of the present invention is comprised of titanium or a titanium alloy.

In accordance with another aspect of the present invention, there is provided a method for treating spinal tumor patient that comprises securing a vertebral stabilizing device to the vertebral bone of the patient by way of a bone anchor comprised of a radiotranslucent material, such anchor having an anchoring portion suitable for embedment into bone and a head portion suitable for a direct or indirect coupling to the stabilizing device.

Accordingly, the present invention advantageously provides for a new and improved anchoring mechanism such as a pedicle screw for implantable spinal support devices that eliminates unacceptable levels of radiotranslucency and radiation scatter while withstanding the tremendous compressive forces transferred from the spine once the device is implanted in a patient. Moreover, the present invention advantageously provides a new treatment method and novel anchoring mechanism for patients being treated for spinal tumors using radiotherapy.

Other objects, features and advantages of the present invention will be apparent to those of ordinary skill in the art in view of the following detailed description of the invention and accompanying drawings.

### BRIEF DESCRIPTION OF THE DRAWINGS

The drawings as provided for herein set forth one exemplary embodiment of the present invention, the detailed description of which follows hereinbelow. The drawings are merely exemplary and are clearly not intended to limit the invention as encompassed by the claims appended herewith.

FIG. 1 is a side view of an exemplary embodiment of a bone anchor as contemplated by the present invention, in this case a pedicle screw.

FIG. 2 is an enlarged side view of the screw head of the pedicle screw as seen in FIG. 1.

FIG. 3 is a side view of the pedicle screw as seen in FIG. 1 coupled to a receiving saddle that functions to operatively couple the screw to a vertebral stabilizing device.

FIG. 4 is a sectional view across line A-A of the pedicle screw as seen in FIG. 3.

FIG. 5 is a perspective side view of the pedicle screw as seen in FIG. 1 coupled to a receiving saddle that functions to operatively couple the screw to a vertebral stabilizing device.

### DETAILED DESCRIPTION OF THE INVENTION

In accordance the present invention, there is provided a bone anchoring mechanism/bone anchor comprised of a radiotranslucent material for securing vertebral stabilizing implants to vertebral bone. Vertebral stabilizing implants/devices are well known in the art of medicine and include but are not limited to spinal rod assemblies and apparatus, spinal cage assemblies and apparatus, etc. that will be readily apparent and available to the artisan.

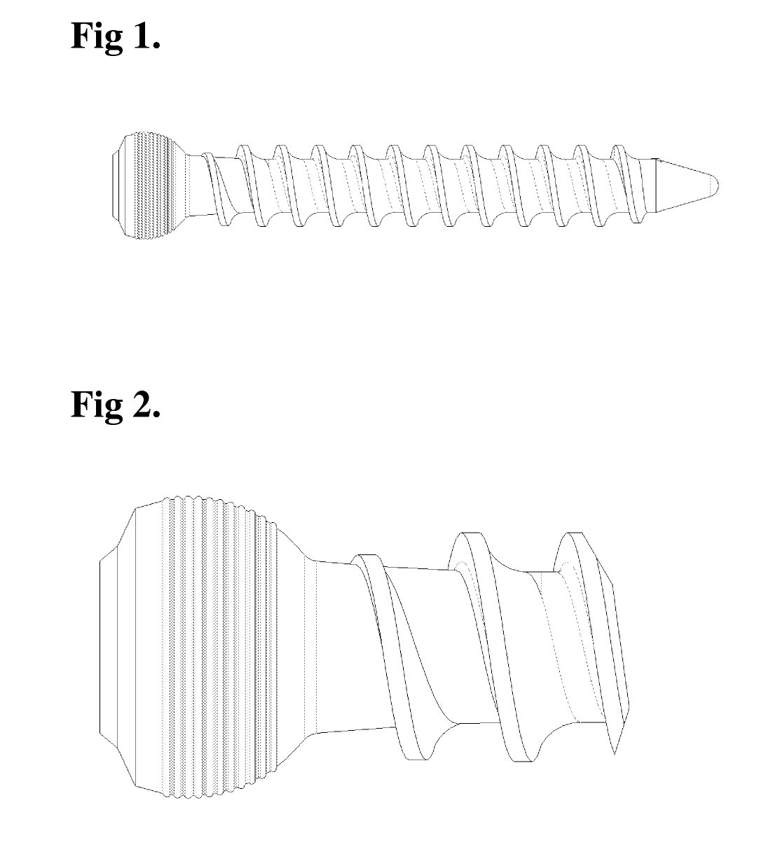

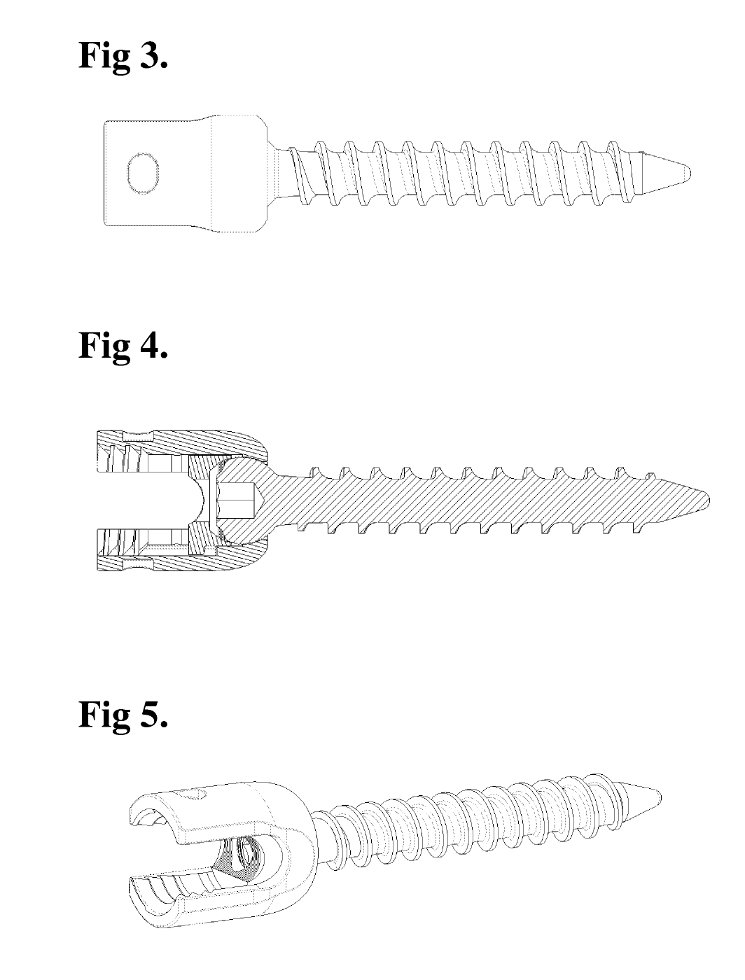

The bone anchoring mechanism of the present invention includes an anchoring portion that is suitable to be embedded into vertebral bone as well as a head portion that can be coupled directly or indirectly to the stabilizing device (see FIGS. 1-4). For instances of indirect coupling, a separate coupling mechanism such as a “saddle”, “tulip” or other connector is employed to communicate with and receive some structural aspect of the stabilizing device be it a rod, rod assembly, plate, cage, etc. (see FIGS. 1-4).

In one preferred, exemplary embodiment, the bone anchor is fashioned in the form of is a pedicle screw, however, other anchors such as barbs, spikes, etc. are known in the art (see for example, U.S. Pat. Nos. 4,834,757 and 4,878,915 to Brantigan). As will be appreciated by those skilled in the art, pedicle screw configurations may selected from various well known designs such as those disclosed in the following patents so long as the design is conducive for manufacture utilizing the radiotranslucent material contemplated by the present invention (see for example, U.S. Pat. Nos. 7,445,627, 7,163,539, 6,858,030, 6,840,940, 6,565,567, 6,554,834, 6,488,681, 6,485,494, 6,402,752, 6,368,319, 6,183,472, 6,063,089, 5,725,528, 5,207,678, 4,946,458, and 4,887,596, all of which are incorporated herewith by reference thereto).

As mentioned above, the bone anchor of the present invention includes an anchoring portion that is suitable to be embedded into vertebral bone as well as a head portion that can be coupled directly or indirectly to the stabilizing device (see FIGS. 1-4). In one embodiment of the present invention, the entire anchor (both anchor portion and head portion) is formed from a radiotranslucent material. In an alternative embodiment, only the anchoring portion is formed from the radiotranslucent material while the head portion is fashioned from stainless steel, titanium, titanium alloy or other appropriate material.

Importantly, it is an essential element of the present invention that the anchoring portion of the bone anchor be comprised of radiotranslucent material as the anchoring site typically occurs in vertebral bone that is in the proximity of the irradiation site during spinal radiosurgery. In instances where it is possible, the entire bone anchor as well as the spinal stabilizing device and any couplings placed therebetween will all be comprised of the radiotranslucent material so as to optimize elimination or reduction of any radiological image obstructions and radiation scatter.

It will be appreciated by those skilled in the art that the term “radiotranslucent material” as used herein is intended to mean any biocompatible material that either eliminates radiopacity and radiation scatter or, alternatively, renders insignificant interference levels of the same relative to radiological imaging obstructions and/or radiation dosage delivery utilizing radiosurgery therapy. The term “biocompatible material” is intended to mean any material that will not elicit a biological response in the patient that results in either the erosion, corrosion, or rejection of the material or that induces an otherwise unfavorable pathological tissue response or toxicity issue.

Accordingly, the radiotranslucent material as contemplated by the present invention typically comprises a medical grade, radiotranslucent polymer or co-polymer that is capable of forming a rigid, loadbearing matrix sufficient to handle the compressive and shear forces transferred to it by a vertebral stabilizing device that has been affixed to a patient's spine. Such medical grade polymer materials are well known and readily available to the art of medical design engineering and include but are not limited to thermosetting polymers, thermoplastic polymers, and mixtures thereof. Moreover, it will be further apparent to those skilled in the art that selection of such polymers or copolymers should be such that the resulting polymeric matrix formed thereby is capable of withstanding the compressive and shear forces mentioned above.

In a further embodiment, the radiotranslucent polymer or copolymer is comprised of at least one of the polymeric materials selected from the following group: poly-ether-ether-ketone (PEEK), poly-ether-ketone-ketone (PEKK), poly-methylmethacrylate (PMMA), polysulfone, polylactide (PLA), poly-L-lactide (PLLA), and poly(glycolic acid) (PGA). Those skilled in the art will appreciate that some instances a ketone-based polymer such as poly-ether-ether-ketone (PEEK) or poly-ether-ketone-ketone (PEKK) would be preferred.

In another exemplary embodiment, the radiotranslucent material comprises a composite substance that is in turn comprised of a medical grade fiber material dispersed within one of the polymeric matrices as described above. As used herein, a composite material is one formed from two or more materials that exhibit performance characteristics exceeding that of the individual components alone.

Medical grade fiber materials are well known to the art and are available in a variety of weights, thickness, and composition. It will be readily appreciated by those skilled in the art that the selection of fiber material as well as its thickness, length, weight, density, etc. must be such that the fibers do not negatively impact the radiotranslucency and radiation scatter requirements of the bone anchors of the present invention to any significant degree. Importantly, the selection of the fiber material should also entertain the appropriate compressive and shear strength considerations that will be readily discernable to the medical device engineer.

In one embodiment of the composite of the present invention, the fiber material is comprised of a least one of either carbon fibers or polyamide fibers. It will be further appreciated by medical design engineers skilled in the art that such fibers are available in different weights, lengths, and densities, the selection of which ultimately affects the rigidity and strength of the resulting anchor in which they are incorporated.

The resulting composite material as contemplated for use in the present invention is a function of i) the type and volume of polymeric material employed ii) the type and volume of fiber material employed (iii) the length and diameter of the fibers, and iv) the orientation of the fibers within the polymer. Variations on these parameters as well as their resulting effects on the end product are well known in the art of medical design engineering. For example, a variation in the choice of fiber type, fiber orientation, fiber diameter, fiber length, and fiber layering will depend on the particular requirements for the anchor. If greater rigidity is required, then either a greater fiber diameter and/or length should be employed or the layering of the fibers should be increased, or both. Conversely, if less rigidity is required, then the volume of the fibers and the layering associated therewith can be reduced.

Notably, the length of the fiber generally varies directly with the strength of the device (although, once the fibers reach a critical length, the strength remains the same). The fibers employed in the composite material of the present invention are typically discontinuous or short fibers, particularly those having a length of less than one millimeter. They are typically considered isotropic with strength and rigidity distributed in more than one direction. Ball bearings or other small parts typically benefit from short fiber construction.

Once the appropriate materials are selected, the composite material may be formed by any one of several well accepted methods known in the art including but not limited to net compression molding via composite pre-peg tape placement, pultrusion, filament winding, braiding, and injection molding.

In yet another embodiment of the present invention, the bone anchor is fashioned in the form of a pedicle screw having the anchoring portion manufactured from a radiotranslucent composite material comprised substantially of short strand carbon fibers disposed within poly-ether-ether-ketone (PEEK) and a head portion comprised substantially from stainless steel, titanium, or a titanium alloy. It should be appreciated that the term “substantially” as used herein is intended to mean that there no statistically significant materials present in the composite other than those mentioned.

In accordance with another aspect of the present invention, there is also provided a method for treating a spinal tumor patient that comprises securing a vertebral stabilizing device to the vertebral bone of the patient by way of an anchoring member comprised of a radiotranslucent material and having an anchoring portion suitable for embedment into bone and a head portion suitable for a direct or indirect coupling to the stabilizing device.

The present invention also provides for a bone anchoring mechanism/bone anchor for securing a vertebral stabilizing device to vertebral bone of a spine tumor patient, the anchor having an anchoring portion suitable for embedment into bone and a head portion suitable for a direct or indirect coupling to the stabilizing device, the orthopedic anchor being comprised of a radiotranslucent material. It is most notably fashioned as a pedicle screw substantially comprised of a composite material that is in turn substantially comprised of short strand carbon fibers disposed within poly-ether-ether-ketone (PEEK).

The foregoing is provided for purposes of illustrating, explaining, and describing various exemplary embodiments of this invention. Modifications and adaptations to these embodiments will be apparent to those skilled in the art and may be made without departing from the scope or spirit of this invention, the nature of which is set forth below in the appended claims.

What is claimed is:

1. A bone anchor for securing a vertebral stabilizing device to vertebral bone, the anchor having an anchoring portion suitable for embedment into bone and a head portion suitable for a direct or indirect coupling to the stabilizing device, the anchoring portion comprised of a radiotranslucent material.

2. The bone anchor of claim 1, wherein the bone anchor is a pedicle screw.

3. The bone anchor of claim 1, wherein the radiotranslucent material is comprised of a radiotranslucent medical grade polymer or co-polymer.

4. The bone anchor of claim 3, wherein the polymer or co-polymer is comprised of at least one of the polymeric materials selected from the group consisting of poly-ether-ether-ketone (PEEK), poly-ether-ketone-ketone (PEKK), poly-methylmethacrylate (PMMA), polysulfone, polylactide (PLA), poly-L-lactide (PLLA), and poly (glycolic acid) (PGA).

5. The bone anchor of claim 1, wherein the radiotranslucent material is comprised of a composite.

6. The bone anchor of claim 5, wherein the composite comprises at least one medical grade fiber material embedded in at least one medical grade polymer or co-polymer.

7. The bone anchor of claim 6, wherein the polymer or co-polymer is comprised of at least one of the polymeric materials selected from the group consisting of poly-ether-ether-ketone (PEEK), poly-ether-ketone-ketone (PEKK), poly-methylmethacrylate (PMMA), polysulfone, polylactide (PLA), poly-L-lactide (PLLA), poly(glycolic acid) (PGA)

8. The bone anchor of claim 7, wherein the polymer or co-polymer is substantially comprised of poly-ether-ether-ketone (PEEK).

9. The bone anchor of claim 6, wherein the fiber material comprises at least one of either carbon fibers or polyamide fibers.

10. The bone anchor of claim 9, wherein the fiber material substantially comprises carbon fibers.

11. The bone anchor of claim 10, wherein the carbon fibers are substantially short strand fibers.

12. The bone anchor of claim 10, wherein the carbon fibers are substantially short strand fibers and the medical grade polymer or co-polymer is comprised of at least one of the following polymeric materials: poly-ether-ether-ketone (PEEK), poly-ether-ketone-ketone (PEKK), polymethyl-methacrylate (PMMA), polysulfone, polylactide (PLA), poly-L-lactide (PLLA), poly(glycolic acid) (PGA).

13. The bone anchor of claim 12, wherein the fiber material comprises short strand carbon fibers and the medical grade polymer or co-polymer comprises poly-ether-ether-ketone (PEEK).

14. The bone anchor of claim 13, wherein the anchor is a pedicle screw.

15. The pedicle screw of claim 14 substantially comprised of short strand carbon fibers disposed within poly-ether-ether-ketone (PEEK).

16. The pedicle screw of claim 15 wherein the anchoring portion is substantially comprised of the radiotranslucent material and the head portion is comprised substantially of at least one material selected from the group consisting of stainless steel, titanium, or titanium alloy.

17. A method of treating a spinal tumor patient that comprises securing a vertebral stabilizing device to the vertebral bone of the patient by way of a bone anchor comprised of a radiotranslucent material, the anchor having an anchoring portion suitable for embedment into bone and a head portion suitable for a direct or indirect coupling to the stabilizing device.

18. The method of claim 17 further comprising selecting the bone anchor from a pedicle screw design comprised of a composite material comprising short strand carbon fibers and poly-ether-ether-ketone (PEEK).

19. An bone anchor for securing a vertebral stabilizing device to vertebral bone of a spine tumor patient, the anchor having an anchoring portion suitable for embedment into bone and a head portion suitable for a direct or indirect coupling to the stabilizing device, wherein the bone anchor is comprised of a radiotranslucent material.

20. The bone anchor of claim 19, wherein the anchor is a pedicle screw comprised of a composite material that is substantially comprised of short strand carbon fibers disposed within poly-ether-ether-ketone (PEEK).
